(19) United States
(12) Patent Application Publication
(10) Pub. No.: US 2008/0045547 A1
(43) Pub. Date: Feb. 21, 2008

(54) Salts And Co-Crystals of Pyrazolopyrimidine Compounds, Compositions Thereof And Methods For Their Production And Use

(76) Inventors: Arnold S. LIPPA, New York, NY (US); Zhengming CHEN, Belle Mead, NJ (US); Phil SKOLNICK, Edgewater, NJ (US)

Correspondence Address: BLACK LOWE & GRAHAM PLLC, Suite 4800, 701 Fifth Avenue, Seattle, WA, 98104, US

(21) Appl. No.: 11/769,628

(22) Filed: Jun. 27, 2007

Related U.S. Application Data
(60) Provisional application No. 60/819,449, filed on Jul. 7, 2006.

(51) Int. Cl. A61K 31/519; A61P 25/00; A61P 25/08; C07D 487/04

(52) U.S. Cl. 514/259.3; 546/112

(57) ABSTRACT

The invention provides pharmaceutically acceptable salts and co-crystals of pyrazolopyrimidine compounds such as zaleplon, indiplon and ocinaplon, processes for their preparation, compositions comprising such salts and co-crystals and methods of using such salts and co-crystals for treating various diseases and conditions.

### RELATED APPLICATIONS

This application claims all priority benefits of U.S. Provisional patent application number 60/819,449, filed Jul. 7, 2006 which is incorporated herein by reference.

### BACKGROUND OF THE INVENTION

Zaleplon, indiplon and ocinaplon belong to the pyrazolopyrimidine class of compounds. Zaleplon, whose chemical name is N-[3-(3-cyanopyrazolo[1,5-a]pyrimidin-7-yl)phenyl]-N-ethylacetamide, has the empirical formula C₁₇H₁₅N₅O and a molecular weight of 305.34. Indiplon, whose chemical name is N-methyl-N-{3-[3-(thiophene-2-carbonyl)-pyrazolo[1,5-a]pyrimidin-7-yl]-phenyl}acetamide, has the empirical formula C₂₀H₁₆N₄O₂S and a molecular weight of 376.43. Ocinaplon, whose chemical name is pyridin-2-yl(7-(pyridin-4-yl)pyrazolo[1,5-a]pyrimidin-3-yl)methanone, has the empirical formula C₁₇H₁₁N₅O and a molecular weight of 301.3. The structures of zaleplon, indiplon and ocinaplon are as follows:

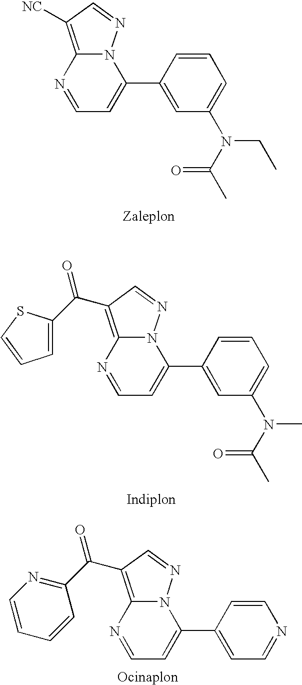

The use of pyrazolopyrimidines as medicinal agents has been described (See, for example, U.S. Pat. Nos. 4,521,422, 6,399,621 and 6,384,221). These compounds exert their pharmacological actions through a family of hetero-oligomeric ligand-gated ion channels commonly referred to as the GABA_{A} receptors (Vanover, K. E. et al., Exp. Clin. Psychopharmacol. 2:223-233, 1994; Damgen, K. and Luddens, H., Neurosci. Res. Commun. 25:139-148, 1999; Sanna, E. et al, Eur. J. Pharmacol. 451:103-110, 2002; Petroski, R. E. et al., J. of Pharmacol. Exp. Ther. 317:369-377, 2006; Lippa, A. et al., Proc. Natl. Acad. Sci. USA 102:7380-7385, 2005; Barnard, E. A. et al., Pharmacol. Rev. 50:291-313, 1998).

Although these compounds all target the same family of ligand-gated ion channels, various substitutions on the pyrazolopyrimidine ring structure can impart major differences in the pharmacological actions (activities) of pyrazolopyrimidines. Thus, zaleplon (Sonata®) has been used for over a decade for the treatment of insomnia and related sleep disorders, and the use of indiplon in treating sleep disorders has been described in several clinical studies (Roth, T. et al., Sleep 26:A87, 2003; Jochelson, P. et al., Sleep 26:A85, 2003). In contrast, ocinaplon has been described as an “anxioselective agent” because it produces anti-anxiety (anxiolytic) actions in humans, but appears to lack a prominent sedative/hypnotic effect common to zaleplon and indiplon (Lippa, et al., Proc. Natl. Acad. Sci. USA 102:7380-7385, 2005). As described in the patent and scientific literature and practiced commercially, these pyrazolopyrimidines are commonly administered in a variety of pharmaceutical formulations as the free base.

### SUMMARY OF EXEMPLARY EMBODIMENTS OF THE INVENTION

While it is evident that pyrazolopyrimidines such as zaleplon, indiplon and ocinaplon are useful as medicinal agents, various improvements in pharmaceutical properties of these compounds are desirable. Such improvements within the invention include, but are not limited to: 1) decreased inter- and intra-subject variability in blood levels (e.g., to provide greater certainty that a given dose will be effective/produce the desired effect); 2) better absorption (often measured as increases in plasma concentrations summed over time, known as the “area under the curve” or “AUC”) that could result in administration of lower doses of drug to achieve satisfactory therapeutic effect and/or reduce side effects; 3) higher maximum plasma concentrations (Cmax) that could result in lower doses of drug required to produce a satisfactory therapeutic effect and/or reduce side effects; 4) decreased time to peak drug concentrations in plasma (often referred to as the tₘₐₓ); and 5) changes in the plasma half life of a compound (often referred to as the t_{1/2}). For example, in the case of a hypnotic agent, where a rapid onset of action and a short duration of action are often viewed as advantageous (in the latter case, to preclude residual drug producing a “hangover” effect upon awakening), achieving objectives 4 and/or 5 above would be viewed as particularly desirable.

Accordingly, it is an object of the present invention to provide new pharmaceutically acceptable salts and co-crystals of compounds within the pyrazolopyrimidine class of compounds.

It is a further object of the present invention to provide compositions comprising pharmaceutically acceptable salts and co-crystals of compounds within the pyrazolopyrimidine class of compounds.

It is an additional object of the present invention to provide forms of pyrazolopyrimidine compounds having improved characteristics, including improved pharmaceutical properties and features as noted above.

It is another object of the present invention to provide processes for the production of pharmaceutically acceptable salts and co-crystals of compounds within the pyrazolopyrimidine class of compounds.

It is an additional object of the present invention to provide methods for the use of pharmaceutically acceptable salts and co-crystals of compounds within the pyrazolopyrimidine class of compounds.

The present invention fulfills these needs and satisfies additional objects and advantages by providing novel, pharmaceutically acceptable salts and co-crystals of compounds within the pyrazolopyrimidine class of compounds.

In exemplary embodiments, the present invention provides pharmaceutically acceptable salts and co-crystals of a compound of the pyrazolopyrimidine class, wherein the compound is N-[3-(3-cyanopyrazolo[1,5-a]pyrimidin-7-yl)phenyl]-N-ethylacetamide (zaleplon), N-methyl-N-{3-[3-(thiophene-2-carbonyl)-pyrazolo[1,5-a]pyrimidin-7-yl]-phenyl}acetamide (indiplon) or pyridin-2-yl(7-(pyridin-4-yl)pyrazolo[1,5-a]pyrimidin-3-yl)methanone (ocinaplon).

The present invention also provides compositions comprising pharmaceutically acceptable salts and co-crystals of a compound of the pyrazolopyrimidine class, wherein the compound is N-[3-(3-cyanopyrazolo[1,5-a]pyrimidin-7-yl)phenyl]-N-ethylacetamide (zaleplon), N-Methyl-N-{3-[3-(thiophene-2-carbonyl)-pyrazolo[1,5-a]pyrimidin-7-yl]-phenyl}acetamide (indiplon) or pyridin-2-yl(7-(pyridin-4-yl)pyrazolo[1,5-a]pyrimidin-3-yl)methanone (ocinaplon).

The present invention further provides methods for preparing pharmaceutically acceptable salts and co-crystals of a compound of the pyrazolopyrimidine class, wherein the compound is N-[3-(3-cyanopyrazolo[1,5-a]pyrimidin-7-yl)phenyl]-N-ethylacetamide (zaleplon), N-Methyl-N-{3-[3-(Thiophene-2-carbonyl)-pyrazolo[1,5-a]pyrimidin-7-yl]-phenyl}acetamide (indiplon) or pyridin-2-yl(7-(pyridin-4-yl)pyrazolo[1,5-a]pyrimidin-3-yl)methanone (ocinaplon), comprising reacting the compound with a salt-forming acid.

The present invention additionally provides methods for preventing or treating a disease or condition amenable to treatment or beneficial intervention using a pyrazolopyrimidine salt or co-crystal of the present invention. In certain aspects of the invention, methods of treatment are provided for various diseases and conditions of the central nervous system in mammals. Examples of such diseases and conditions of the central nervous system amenable to treatment using pyrazolopyrimidine salts and co-crystals of the present invention include, but are not limited to, insomnia and other sleep disorders, anxiety disorders such as general anxiety disorder and panic disorders, muscle spasms, tinnitus, pain, acute psychosis, including acute psychotic episodes, epilepsy and other seizure disorders. In exemplary embodiments, the invention provides methods for preventing or treating a disease or condition selected from the group consisting of a sleep disorder, an anxiety disorder, a seizure disorder, muscle spasms, and tinnitus, comprising administering to a mammalian subject in need of such prevention or treatment an effective amount of a pharmaceutically acceptable salt or co-crystal of a compound of the pyrazolopyrimidine class, wherein the compound is selected from N-[3-(3-cyanopyrazolo[1,5-a]pyrimidin-7-yl)phenyl]-N-ethylacetamide (zaleplon), N-methyl-N-{3-[3-(thiophene-2-carbonyl)-pyrazolo[1,5-a]pyrimidin-7-yl]-phenyl}acetamide (indiplon) or pyridin-2-yl(7-(pyridin-4-yl)pyrazolo[1,5-a]pyrimidin-3-yl)methanone (ocinaplon).

### BRIEF DESCRIPTION OF THE DRAWINGS

FIG. 1 shows differential scanning calorimetry profiles for (a) zaleplon free base; (b) zaleplon phosphate; (c) zaleplon sulfate; (d) zaleplon hydrochloride; and (e) zaleplon hydrobromide.

### DETAILED DESCRIPTION OF EXEMPLARY EMBODIMENTS OF THE INVENTION

The present invention provides pharmaceutically acceptable salts and co-crystals of a compound of the pyrazolopyrimidine class.

In particular, the present invention provides pharmaceutically acceptable salts and co-crystals of a compound of the pyrazolopyrimidine class, wherein the compound is N-[3-(3-cyanopyrazolo[1,5-a]pyrimidin-7-yl)phenyl]-N-ethylacetamide (zaleplon), N-Methyl-N-{3-[3-(Thiophene-2-carbonyl)-pyrazolo[1,5-a]pyrimidin-7-yl]-phenyl}acetamide (indiplon) or pyridin-2-yl(7-(pyridin-4-yl)pyrazolo[1,5-a]pyrimidin-3-yl)methanone (ocinaplon).

The present invention also provides pharmaceutically acceptable salts and co-crystals of a compound of the pyrazolopyrimidine class, wherein the compound is N-[3-(3-cyanopyrazolo[1,5-a]pyrimidin-7-yl)phenyl]-N-ethylacetamide (zaleplon), N-Methyl-N-{3-[3-(Thiophene-2-carbonyl)-pyrazolo[1,5-a]pyrimidin-7-yl]-phenyl}acetamide (indiplon) or pyridin-2-yl(7-(pyridin-4-yl)pyrazolo[1,5-a]pyrimidin-3-yl)methanone (ocinaplon), having improved pharmacokinetic properties when compared to the free base. Such improvements include but are not limited to: 1) decreased inter- and intra-subject variability in blood levels (e.g., to provide greater certainty that a given dose will be effective/produce the desired effect); 2) better absorption (often measured as increases in plasma concentrations summed over time, known as the “area under the curve” or “AUC”) that could result in administration of lower doses of drug to achieve satisfactory therapeutic effect and/or reduce side effects; 3) higher maximum plasma concentrations (Cmax) that could result in lower doses of drug required to produce a satisfactory therapeutic effect and/or reduce side effects; 4) decreased time to peak drug concentrations in plasma (often referred to as the tₘₐₓ); and 5) changes in the plasma half life of a compound (often referred to as the t_{1/2}).

In certain embodiments, the improved pharmacokinetic property of a pyrazolopyrimidine salt or co-crystal of the invention is better absorption, as evidenced by, e.g., an increase in plasma concentration summed over time. In exemplary embodiments, an increase in plasma concentration summed over time of a pyrazolopyrimidine salt or co-crystal of the invention is from about 10% to about 20%, about 20% to about 50%, about 50% to about 100%, about 100% to about 200%, about 200% to about 500%, about 500% to 1000%, or greater, when compared to plasma concentration summed over time exhibited by the corresponding free base.

In other exemplary embodiments, the improved pharmacokinetic property is a higher maximum plasma concentration, wherein the increase in maximum plasma concentration of the pyrazolopyrimidine salt or co-crystal is from about 10% to about 20%, about 20% to about 50%, about 50% to about 100%, about 100% to about 200%, about 200% to about 500%, or about 500% to 1000%, or greater, when compared to the maximum plasma concentration exhibited by the free base.

In other exemplary embodiments, the improved pharmacokinetic characteristic is decreased time to peak drug concentration in plasma, wherein the time to peak drug concentration in plasma of the pyrazolopyrimidine salt or co-crystal is about 10% to about 20%, about 20% to about 50%, about 50% to about 75%, or about 75% to about 99% of the time to peak drug concentration in plasma exhibited by the free base.

In other exemplary embodiments, the improved phanrnacokinetic property is a change in plasma half-life, wherein the plasma half-life of the pyrazolopyiimidine salt or co-crystal is increased or decreased by about 10% to about 20%, about 20% to about 50%, about 50% to about 75%, or about 75% to about 99% compared to the plasma half-life exhibited by the free base.

In yet additional exemplary embodiments, the improved pharmacokinetic characteristic is decreased inter- and/or intra-subject variability in blood levels, e.g., as evidenced by decreased inter- and/or intra-subject variability in plasma concentration summed over time, wherein the inter- and/or intra-subject variability in plasma concentration summed over time of the pharmaceutically acceptable salt or co-crystal of a compound of the pyrazolopyrimidine class is about 10% to about 20%, about 20% to about 50%, about 50% to about 75%, or about 75% to about 99% compared to the inter- and/or intra-subject variability in plasma concentration summed over time exhibited by the free base.

Pharmaceutically acceptable salts and co-crystals of the present invention include suitable acid addition salts and co-crystals of a compound of the pyrazolopyrimidine class formed from acids that fonn non-toxic salts, examples of which are hydrochloride, hydrobromide, hydroiodide, sulphate, hydrogen sulphate, nitrate, phosphate, and hydrogen phosphate salts and saccharin co-crystals. Additional examples of phanraceutically acceptable addition salts include both inorganic and organic acid addition salts. The phamnaceutically acceptable salts and co-crystals include, but are not limited, organic acid salts such as acetate, citrate, lactate, succinate, tartrate, maleate, fumarate, mandelate, acetate, dichloroacetate, trifluoroacetate, oxalate, fonnate and the like; sulfonates such as methanesulfonate, benzenesulfonate, p-toluenesulfonate and the like; and amino acid salts such as arginate, asparginate, glutamate, tartrate, gluconate and the like. In a particular embodiment, the pharmaceutically acceptable salt or co-crystal is the hydrobromide, hydrochloride, sulfate or phosphate salt of N-[3-(3-cyanopyrazolo[1,5-a]pyrimidin-7-yl)phenyl]-N-ethylacetamide (zaleplon). In an additional embodiment, the pharmaceutically acceptable salt or co-crystal is the hydrobromide, hydrochloiide, sulfate or phosphate salt of N-methyl-N-{3-[3-(thiophene-2-carbonyl)-pyrazolo[1,5-a]pyrimidin-7-yl]-phenyl}acetamide (indiplon). In a further embodiment, the pharmaceutically acceptable salt or co-crystal is the hydrobromide, hydrochloride, sulfate or phosphate salt of pyridin-2-yl(7-(pyridin-4-yl)pyrazolo[1,5-a]pyrimidin-3-yl)methanone (ocinaplon). In another embodiment, the pharmaceutically acceptable salt or co-crystal is the dihydrobromide, dihydrochloride, disulfate or diphosphate salt of pyridin-2-yl(7-(pyridin-4-yl)pyrazolo[1,5-a]pyrimidin-3-yl)methanone (ocinaplon).

It should be understood that pharmaceutically acceptable salt or co-crystal of derivatives and analogs of N-[3-(3-cyanopyrazolo[1,5-a]pyrimidin-7-yl)phenyl]-N-ethylacetamide (zaleplon), N-methyl-N-{3-[3-(thiophene-2-carbonyl)-pyrazolo[1,5-a]pyrimidin-7-yl]-phenyl}acetamide (indiplon) or pyridin-2-yl(7-(pyridin-4-yl)pyrazolo[1,5-a]pyrimidin-3-yl)methanone (ocinaplon) can also be prepared and are included within the present invention.

The present invention also provides methods for preparing a pharmaceutically acceptable salt or co-crystal of a compound of the pyrazolopyrimidine class.

In certain embodiments, the present invention provides methods for preparing a pharmaceutically acceptable salt or co-crystal of a compound of the pyrazolopyrimidine class, wherein the compound is N-[3-(3-cyanopyrazolo[1,5-a]pyrimidin-7-yl)phenyl]-N-ethylacetamide (zaleplon), N-methyl-N-{3-[3-(thiophene-2-carbonyl)-pyrazolo[1,5-a]pyrimidin-7-yl]-phenyl}acetamide (indiplon) or pyridin-2-yl(7-(pyridin-4-yl)pyrazolo[1,5-a]pyrimidin-3-yl)methanone (ocinaplon), comprising reacting the compound with a salt-forming acid.

In producing a pharmaceutically acceptable salt or co-crystal of a compound of the pyrazolopyrimidine class in accordance with the methods of the present invention, the compound of the pyrazolopyrimidine class dissolved in an appropriate solvent or solvent mixture can be reacted with a salt-forming acid.

Various forms of the compound of the pyrazolopyrimidine class can be utilized as the starting material. For example, racemates, enantiomers, stereoisomers, solvates, hydrates and polymorphs of the pyrazolopyrimidine compound can be used. With respect to the solvent, any solvent or mixture of solvents can be used which allow, depending on the conditions used, both dissolution of the pyrazolopyrimidine compound in the solvent or solvent mixture and reaction of the pyrazolopyrimidine compound with a salt-forming acid. Suitable solvents include, for example, dichloromethane, ethers such as diethyl ether, ethyl acetate, mixtures thereof, and mixtures thereof with water.

Various procedures may be used to prepare the pyrazolopyrimidine compound starting material used in the methods of the present invention. For example, an exemplary prior synthetic method for producing zaleplon is described in Shen, J. et al., Zhongguo Yiyao Gongye Zazhi 33:313-314, 2002, incorporated by reference herein. Briefly, N-[3(3-cyanopyrazolo[1,5-a]pyrimidin-7-yl)phenyl]-N-ethyl-acetamide] (zaleplon) is prepared by the cyclocondensation reaction of 3-dimethylamino-1-(3-N-ethyl-N-acetylaminophenyl)-2-propen-1-one with 3-aminopyrazole-4-carbonitrile in an acidic solvent. The 3-dimethylamino-1-(3-N-ethyl-N-acetylaminophenyl)-2-propen-1-one is prepared from 3-aminoacetophenone via intermediate 3-[3-(dimethylamino)-2-propenoyl]acetanilide followed by ethylation.

Exemplary prior synthetic methods for producing indiplon are described in Example 6 below and in U.S. Patent Application Publication, Publication No. 2002/0198221, incorporated by reference herein.

An exemplary prior synthetic method for producing ocinaplon is described in U.S. Pat. No. 4,521,422, incorporated by reference herein. Another exemplary synthetic scheme for producing ociniplon is as follows: 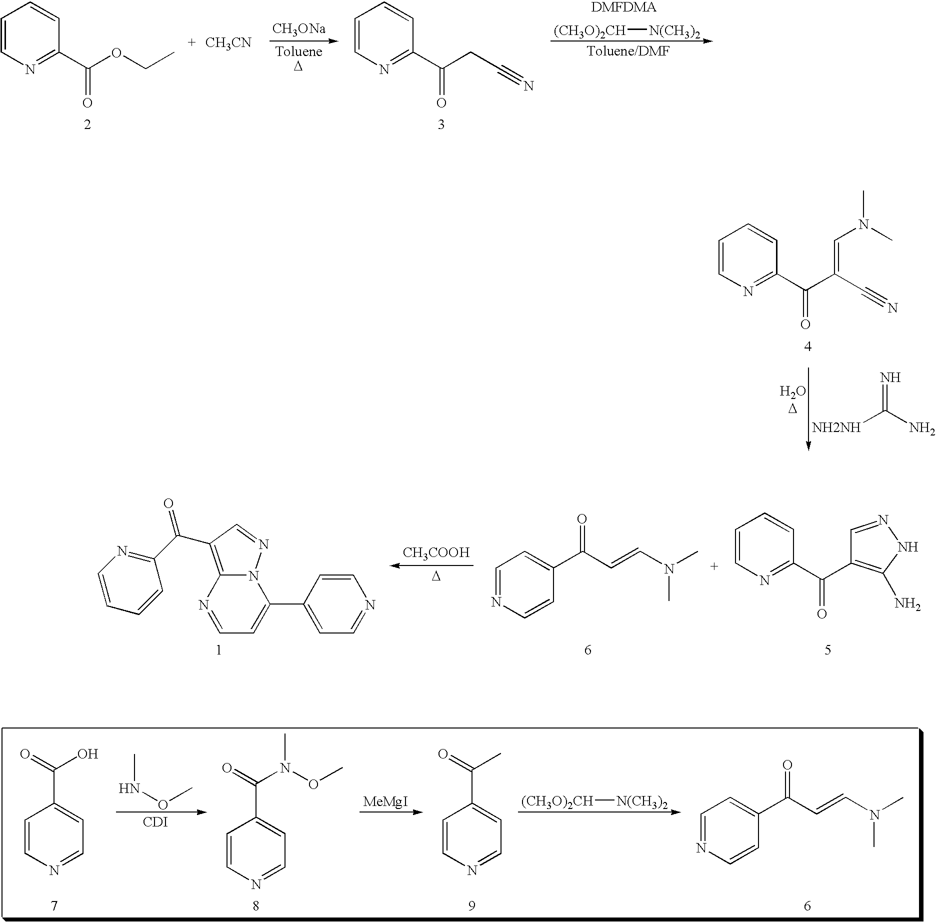
In the foregoing synthetic scheme, 2-ethylpicolinate is converted into pyridylcarbonylacetonitrile, which then reacts with dimethylformamide dimethylacetal (DMFDMA) to form compound 4. Compound 4 reacts with aminoguanidine to afford compound 5. Compound 6 is prepared from 4-pyridylcarboxylic acid via 4-acetylpyridine. The condensation of the aminopyrazole 5 with compound 6 provide ocinaplon 1.

Various salt-forming acids can be used to prepare the pharmaceutically acceptable salts and co-crystals of a compound of the pyrazolopyrimidine class of the present invention. In general, an appropriate salt-forming acid can be chosen depending on the identity of the salt or co-crystal to be formed. For example, hydrochloric acid can be used to form the hydrochloride salt, hydrobromic acid can be used to form the hydrobromide salt, sulfuric acid can be used to form the sulfate salt and phosphoric acid can be used to form the phosphate salt.

Following preparation of a pharmaceutically acceptable salt or co-crystal of a compound of the pyrazolopyrimidine class, various techniques can be used to isolate the pharmaceutically acceptable salt or co-crystal. For example, precipitation followed by a collection technique such as filtration, centrifugation or decantation of the supernatant can be used to isolate the pharmaceutically acceptable salt or co-crystal of a compound of the pyrazolopyrimidine class. In this regard, the pyrazolopyrimidine salt can be precipitated by the addition of a precipitant. A precipitant in this case is defined as a second liquid that is added to a solution to reduce the solubility of the dissolved compound, causing its precipitation and maximizing the yield of product. It is necessary for the original solvent and the added precipitant to be completely miscible with one another in all proportions. Examples of useful precipitants within the scope of the present invention include diethyl ether and ethyl acetate. Optionally, once the precipitate is obtained, it can be washed using a solvent in which the medium is soluble and the precipitate is insoluble. If desired, the precipitate can then be dried to remove any residual solvent. The drying can optionally be performed at reduced pressure to facilitate the removal of any residual solvent.

As used herein, the terms “prevention” and “preventing,” when referring to a disease or condition, refer to a reduction in the risk or likelihood that a mammalian subject will develop the disease or condition, or a reduction in the risk or likelihood of recurrence of the disease or condition once a mammalian subject has been cured, restored to a normal state, or placed in remission from the disease or condition.

As used herein, the terms “treatment” or “treating,” when referring to a disease or condition, refers to inhibiting or reducing the progression or severity of, or delaying the onset of, the disease or condition.

The pyrazolopyrimidine salts and co-crystals of the invention may be utilized in preventing or treating various diseases and conditions of the central nervous system in mammals. Mammalian subjects amenable for treatment with the pyrazolopyrimidine salts and co-crystals of the invention include, but are not limited to, human and other mammalian subjects suffering Prom a disease or condition of the central nervous system that is amenable to treatment or beneficial intervention using the pyrazolopyrimidine salts and co-crystals of the present invention, such as insomnia and other sleep disorders, anxiety disorders such as general anxiety disorder and panic disorders, muscle spasms, tinnitus, pain, acute psychosis, including acute psychotic episodes, and epilepsy and other seizure disorders. In general, the pyrazolopyrimidine salts and co-crystals of the present invention are useful as anxiolytic, sleep-inducing, sedative-hypnotic, anti-convulsant, anti-epileptic, and skeletal muscle relaxant agents.

Administration of an effective amount of a pyrazolopyrimidine salt or co-crystal of the present invention to a mammalian subject presenting with a disease or condition amenable to treatment or beneficial intervention using the pyrazolopyrimidine salts and co-crystals of the present invention, will detectably treat, alleviate, eliminate, or prevent the targeted disease or condition and/or one or more symptom(s) associated therewith. In exemplary embodiments, administration of a pyrazolopyrimidine salt or co-crystal of the present invention to a suitable test subject will yield a reduction in the targeted disease or condition, or one or more targeted symptom(s) associated therewith, by at least 10%, 20%, 30%, 50% or greater, up to a 75-90%, or 95% or greater, reduction in the one or more target symptom(s), compared to placebo-treated or other suitable control subjects. Comparable levels of efficacy are contemplated for the entire range of diseases or conditions described herein for treatment or prevention using the pyrazolopyrimidine salts and co-crystals of the present invention.

It should be understood that a pyrazolopyrimidine salt or co-crystal of the present invention can be combinatorially formulated or coordinately administered with a second therapeutic agent or method—yielding a formulation or method effective to prevent or treat a disease or condition amenable to treatment or beneficial intervention using the pyrazolopyrimidine salts and co-crystals of the present invention, in a mammalian subject. Accordingly, a pyrazolopyrimidine salt or co-crystal of the present invention may be utilized in combinatorial formulations and coordinate administration methods which employ an effective amount of a pyrazolopyrimidine salt or co-crystal of the present invention and one or more additional active agent(s) that is/are combinatorially formulated or coordinately administered with such pyrazolopyrimidine salt or co-crystal to yield a combinatorial formulation or coordinate administration method that is effective to prevent or treat a disease or condition amenable to treatment or beneficial intervention using the pyrazolopyrimidine salts and co-crystals of the present invention, and/or one or more symptom(s) associated therewith, in a mammalian subject.

Additionally, a pyrazolopyrimidine salt or co-crystal of the present invention can be used in combination therapy with at least one other therapeutic agent or method. In this context, a pyrazolopyrimidine salt or co-crystal of the present invention can be administered concurrently or sequentially with administration of a second therapeutic agent, for example a second agent that acts to prevent or treat the same or a different disorder or symptom(s) for which the pyrazolopyrimidine salt or co-crystal of the present invention is administered. The pyrazolopyrimidine salt or co-crystal of the present invention and the second therapeutic agent can be combined in a single composition or administered in different compositions. The coordinate administration may be done simultaneously or sequentially in either order, and there may be a time period when only one or both (or all) active therapeutic agents, individually and/or collectively, exert their biological activities and therapeutic effects. A distinguishing aspect of all such coordinate treatment methods is that the pyrazolopyrimidine salt or co-crystal of the present invention exerts at least some detectable therapeutic activity towards preventing or treating a targeted disease or condition, which may or may not be in conjunction with a secondary clinical response provided by the secondary therapeutic agent. Often, the coordinate administration of a pyrazolopyrimidine salt or co-crystal of the present invention with a secondary therapeutic agent as contemplated herein will yield an enhanced therapeutic response beyond the therapeutic response elicited by either or both the pyrazolopyrimidine salt or co-crystal of the present invention and/or secondary therapeutic agent alone.

Within the combinatorial formulations and methods of the invention, a wide range of useful secondary therapeutic agents are contemplated. In exemplary embodiments, the secondary therapeutic agent will often comprise a second active agent effective for treating the same targeted disease or condition of the central nervous system as treated by the pyrazolopyrimidine salt or co-crystal of the invention. Thus, exemplary secondary therapeutic agents for use within these aspects of the invention include any of a wide selection of known drugs for treating sleep disorders, anxiety disorders such as general anxiety disorder and panic disorders, muscle spasms, tinnitus, pain, acute psychosis, including acute psychotic episodes, epilepsy and other seizure disorders. Often times, the secondary therapeutic agent will be effective in combination with the pyrazolopyrimidine salt or co-crystal of the invention yielding greater therapeutic efficacy and/or lesser adverse side effects than either single agent administered alone at the same or increased, individual dosage levels.

Since a pyrazolopyrimidine salt or co-crystal of the present invention may need to be administered to a patient chronically for the purpose of preventing or treating a particular disease or condition, in one embodiment combination therapy involves alternating between administering a pyrazolopyrimidine salt or co-crystal of the present invention and a second therapeutic agent (i.e., alternating therapy regimens between the two drugs, e.g., at one week, one month, three month, six month, or one year intervals). Alternating drug regimens in this context will often reduce or eliminate adverse side effects, such as toxicity, that may attend long-term administration of one or both drugs alone.

The active pyrazolopyrimidine salts and co-crystals of the present invention may be optionally formulated with a pharmaceutically acceptable carrier and/or various excipients, vehicles, stabilizers, buffers, preservatives, etc. An “effective amount,” “therapeutic amount,” “therapeutically effective amount,” or “effective dose” is an effective amount or dose of an active pyrazolopyrimidine salt or co-crystal as described herein sufficient to elicit a desired pharmacological or therapeutic effect in a mammalian subject—typically resulting in a measurable reduction in an occurrence, frequency, or severity of one or more symptom(s) associated with or caused by the targeted disease or condition, including any combination of symptoms, diseases, conditions, or disorders associated with or caused by the targeted disease or condition, in the subject. In certain embodiments, when a pyrazolopyrimidine salt or co-crystal of the present invention is administered to treat a particular disease or condition, an effective amount of the pyrazolopyrimidine salt or co-crystal will be an amount sufficient in vivo to delay or eliminate onset of symptoms of the targeted disease or condition. Therapeutic efficacy can alternatively be demonstrated by a decrease in the frequency or severity of symptoms associated with the treated disease or condition, or by altering the nature, recurrence, or duration of symptoms associated with the treated disease or condition. Therapeutically effective amounts, and dosage regimens, of the pyrazolopyrimidine salt or co-crystal of the present invention, will be readily determinable by those of ordinary skill in the art, often based on routine clinical or patient-specific factors.

Suitable routes of administration for a pyrazolopyrimidine salt or co-crystal of the present invention include, but are not limited to, oral, buccal, nasal, aerosol, topical, transdermal, mucosal, injectable, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection.

Suitable effective unit dosage amounts of a pyrazolopyrimidine salt or co-crystal of the present invention for mammalian subjects may range from about 1.25 mg to about 400 mg, about 2.5 mg to about 300 mg, about 5.0 mg to about 200 mg, about 10 mg to about 100 mg, or about 15 mg to about 50 mg. In certain embodiments, the effective unit dosage will be selected within narrower ranges of, for example, about 2.5 mg to about 10 mg, about 10 mg to about 50 mg, about 50 mg to about 100 mg, about 100 mg to about 200 mg, about 200 mg to about 300 mg or about 300 mg to about 400 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In exemplary embodiments, dosages of about 2.5 mg to about 10 mg, about 10 mg to about 100 mg, about 100 mg to about 200 mg, about 200 mg to about 300 mg, or about 300 mg to about 400 mg, are administered one, two, three, or four times per day. In other embodiments, dosages of 2.5-10 mg, 10-20 mg, 20-50 mg, 50-200 mg, or 200-400 mg are administered once, twice or three times daily. In alternate embodiments, dosages are calculated based on body weight, and may be administered, for example, in amounts from about 0.05 mg/kg to about 15 mg/kg per day, 0.1 mg/kg to about 12.5 mg/kg per day, 0.25 mg/kg to about 10 mg/kg per day, 0.5 mg/kg to about 7.5 mg/kg per day, 0.75 mg/kg to about 5 mg/kg per day or 1 mg/kg to about 5 mg/kg per day. In farther embodiments, effective unit dosage amounts of a salt or co-crystal of a compound of the pyrazolopyrimidine class, wherein the compound is N-[3-(3-cyanopyrazolo[1,5-a]pyrimidin-7-yl)phenyl]-N-ethylacetamide (zaleplon), may range from about 1.25 mg to about 20 mg. In additional embodiments, effective unit dosage amounts of a salt or co-crystal of a compound of the pyrazolopyrimidine class, wherein the compound is N-methyl-N-{3-[3-(thiophene-2-carbonyl)-pyrazolo[1,5-a]pyrimidin-7-yl]-phenyl}acetamide (indiplon), may range from about 1.25 mg to about 20 mg. In other embodiments, effective unit dosage amounts of a salt or co-crystal of a compound of the pyrazolopyrimidine class, wherein the compound is pyridin-2-yl(7-(pyridin-4-yl)pyrazolo[1,5-a]pyrimidin-3-yl)methanone (ocinaplon), may range from about 60 mg to about 240 mg.

The amount, timing and mode of delivery of compositions comprising an effective amount of a pyrazolopyrimidine salt or co-crystal of the present invention will be routinely adjusted on an individual basis, depending on such factors as weight, age, gender, and condition of the individual, the acuteness of the condition to be treated and/or related symptoms, whether the administration is prophylactic or therapeutic, and on the basis of other factors known to effect drug delivery, absorption, pharmacokinetics, including half-life, and efficacy. An effective dose or multi-dose treatment regimen for a pyrazolopyrimidine salt or co-crystal of the present invention will ordinarily be selected to approximate a minimal dosing regimen that is necessary and sufficient to substantially prevent or alleviate one or more symptom(s) of a targeted disease or condition in the subject, as described herein. Thus, following administration of a pyrazolopyrimidine salt or co-crystal of the present invention, test subjects will exhibit a 10%, 20%, 30%, 50% or greater reduction, up to a 75-90%, or 95% or greater, reduction, in one or more symptoms associated with a targeted disease or condition compared to placebo-treated or other suitable control subjects.

Pharmaceutical dosage fonms of a pyrazolopyrimidine salt or co-crystal of the present invention may optionally include excipients recognized in the art of pharmaceutical compounding as being suitable for the preparation of dosage units as discussed above. Such excipients include, without limitation, binders, fillers, lubricants, emulsifiers, suspending agents, sweeteners, flavorings, preservatives, buffers, wetting agents, disintegrants, effervescent agents and other conventional excipients and additives.

The compositions comprising an effective amount of a pyrazolopyrimidine salt or co-crystal of the present invention can thus include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives and agents will often be biologically inactive and can be administered to patients without causing deleterious side effects or interactions with the active agent.

If desired, a pyrazolopyrimidine salt or co-crystal of the present invention can be administered in a controlled release form by, for example, use of a slow release carrier, such as a hydrophilic, slow release polymer. Exemplary controlled release agents in this context include, but are not limited to, hydroxypropyl methyl cellulose, having a viscosity in the range of about 100 cps to about 100,000 cps.

Formulations comprising an effective amount of a pyrazolopyrimidine salt or co-crystal of the present invention may also include polymers for extended release following parenteral administration. Such polymeric materials are well known to those of ordinary skill in the pharmaceutical compounding arts. Extemporaneous injection solutions, emulsions and suspensions may be prepared from sterile powders, granules and tablets of the kind previously described. Exemplary unit dosage formulations contain a daily dose or unit, daily sub-dose, as described herein above, or an appropriate fraction thereof, of the active ingredient(s).

A pyrazolopyrimidine salt or co-crystal of the present invention may be encapsulated for delivery in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions.

A pyrazolopyrimidine salt or co-crystal of the present invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose, dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms include tablets, which may be prepared by any conventional method of preparing pharmaceutical oral unit dosage forms. Oral unit dosage forms, such as tablets, may contain one or more conventional additional formulation ingredients, including, but not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and in certain embodiments in less than thirty seconds. By effervescent agent is meant a couple, typically an organic acid and a carbonate or bicarbonate. Such rapidly acting dosage forms would be useful, for example, in the prevention or treatment of acute attacks of panic disorder.

The compositions comprising an effective amount of a pyrazolopyrimidine salt or co-crystal of the present invention can be prepared and administered in any of a variety of inhalation or nasal delivery forms known in the art. Devices capable of depositing aerosolized formulations of a pyrazolopyrimidine salt or co-crystal of the present invention in the sinus cavity or pulmonary alveoli of a patient include metered dose inhalers, nebulizers, dry powder generators, sprayers, and the like. Pulmonary delivery to the lungs for rapid transit across the alveolar epithelium into the blood stream may be particularly useful in treating impending episodes of seizures or panic disorder. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of a pyrazolopyrimidine salt or co-crystal of the present invention, and any additional active or inactive ingredient(s).

Intranasal and pulmonary delivery permits the passage of an active pyrazolopyrimidine salt or co-crystal to the blood stream directly after administering an effective amount of the pyrazolopyrimidine salt or co-crystal to the nose or lung. In the case of intranasal delivery, this mode of delivery can achieve direct, or enhanced, delivery of an active pyrazolopyrimidine salt or co-crystal to a mammalian subject.

For intranasal and pulmonary administration, a liquid aerosol formulation will often contain an active pyrazolopyrimidine salt or co-crystal of the present invention combined with a dispersing agent and/or a physiologically acceptable diluent. Alternatively, dry powder aerosol formulations may contain a finely divided solid form of the subject pyrazolopyrimidine salt or co-crystal and a dispersing agent allowing for the ready dispersal of the dry powder particles. With either liquid or dry powder aerosol formulations, the formulation must be aerosolized into small, liquid or solid particles in order to ensure that the aerosolized dose reaches the mucous membranes of the nasal passages or the lung. The term “aerosol particle” is used herein to describe a liquid or solid particle of a sufficiently small particle diameter, e.g., in a range of from about 2-5 microns, suitable for nasal or pulmonary distribution to targeted mucous or alveolar membranes. Other considerations include the construction of the delivery device, additional components in the formulation, and particle characteristics. These aspects of nasal or pulmonary administration of drugs are well known in the art, and manipulation of formulations, aerosolization means, and construction of delivery devices, is within the level of ordinary skill in the art.

Yet additional formulations of a pyrazolopyrimidine salt or co-crystal of the present invention are provided for parenteral administration (e.g., intravenously, intramuscularly, subcutaneously or intraperitoneally), including aqueous and non-aqueous sterile injection solutions which may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The parenteral preparations may be solutions, dispersions or emulsions suitable for such administration. Pharmaceutically acceptable formulations and ingredients will typically be sterile or readily sterilizable, biologically inert, and easily administered. Parenteral preparations typically contain buffering agents and preservatives, and may be lyophilized to be re-constituted at the time of administration. The formulations may be presented in unit-dose or multi-dose containers.

The following examples illustrate certain embodiments of the present invention, and are not to be construed as limiting the present disclosure.

### EXAMPLE 1

### Preparation of Zaleplon Hydrochloride

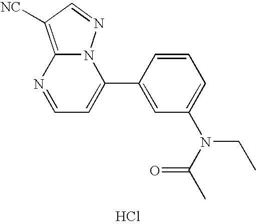

The zaleplon free base used in examples 1-6 and 11 was prepared according to the method of Shen, J. et al., Zhongguo Yiyao Gongye Zazhi 33:313-314, 2002. To prepare zaleplon hydrochloride, zaleplon free base (5 g) was dissolved in anhydrous dichloromethane (12 ml), and then 8 ml of 2N HCl/ether was added dropwise with stirring. The reaction mixture was stirred for 1 h. Diethyl ether (80 ml) was added and the solid was filtered, washed with 150 ml of diethyl ether and dried in the oven. Zaleplon hydrochloride was isolated as a white solid (5.372 g, 98% yield, 100% purity by HPLC).

¹H NMR (DMSO-d₆) δ ppm 11.66-11.93 (broad, 1 H) 8.92 (d, 1 H), 8.86 (s, 1 H), 8.00-8.12 (m, 2 H), 7.55-7.74 (m, 3 H), 3.70 (q, 2 H), 1.74-1.87 (s, 3 H) 1.03 (t, 3 H). ¹³C NMR (DMSO-d₆) δ ppm 168.52, 153.74, 151.04, 147.31, 146.60, 142.63, 131.34, 130.66, 129.92, 129.61, 128.90, 113.40, 110.94, 81.44, 43.10, 22.74, 12.93. Elemental analysis: C 59.4%, N 20.3%, H 4.8%, Cl 10.4%.

### EXAMPLE 2

### Preparation of Zaleplon Hydrobromide

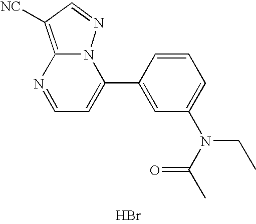

Zaleplon free base (5 g) was suspended in ethyl acetate and dichloromethane was added to solubilize the mixture. 12 ml of 33% HBr/AcOH was then added dropwise with vigorous stirring. The liquid was then discarded and diethyl ether was added to the mixture. This mixture was stirred for 0.5 h, after which the liquid was discarded and the precipitate was washed with ether. The solid was then filtered and dried in vacuo. Zaleplon hydrobromide was isolated as a yellowish solid (5.805 g, 92% yield, 98.4% purity by HPLC).

¹H NMR (DMSO-d₆) δ ppm 10.32 (broad, 1 H), 8.89 (d, J=4.49 Hz, 1 H), 8.83 (s, 1 H), 8.06 (d, J=7.61 Hz, 1 H), 8.00 (s, 1 H), 7.52-7.72 (m, 3 H) 3.67 (q, J=7.09 Hz, 2 H) 1.78 (s, 3 H) 0.96-1.04 (m, 3 H). ¹³C NMR (DMSO-d₆) δ ppm 168.53, 153.75, 151.05, 147.32, 146.60, 142.63, 131.35, 130.66, 129.94, 129.61, 128.92, 113.41, 110.96, 81.44, 43.12, 22.75, 12.94.

Elemental analysis: C 51.7%, N 17.4%, H 4.2%, Br 21.5%.

### EXAMPLE 3

### Preparation of Zaleplon Sulfate

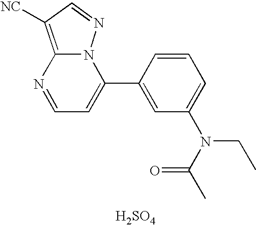

Zaleplon free base (5 g) was dissolved in anhydrous dichloromethane (5 ml), and then 0.34 ml of concentrated sulfuric acid was added dropwise with stirring. The reaction mixture was vortexed and diethyl ether (10 ml) was added to the mixture. The liquid was then discarded and more ether added. This was repeated 3 times. The solid was washed with ether and dried in vacuo. Zaleplon sulfate was isolated as an off-white solid (5.239 g, 78% yield, 97.4% purity by HPLC).

¹H NMR (DMSO-d6) δ ppm 11.02 (broad, 2 H), 8.92 (d, J=4.49 Hz, 1 H), 8.86 (s, 1 H), 8.09 (d, J=7.42 Hz, 1 H), 8.03 (s, 1 H), 7.55-7.75 (m, 3 H), 3.70 (q, J=6.90 Hz, 2 H), 1.81 (s, 3 H), 1.04 (t, J=6.93 Hz, 3 H). 13C NMR (DMSO-d6) δ ppm 169.24, 154.49, 151.78, 148.04, 147.34, 143.36, 132.07, 131.40, 130.66, 130.34, 129.66, 114.15, 111.68, 82.17, 43.85, 23.46, 13.64.

Elemental analysis: C 49.8%, N 17.0%, H 4.3%, S 7.9%.

### EXAMPLE 4

### Preparation of Zaleplon Phosphate

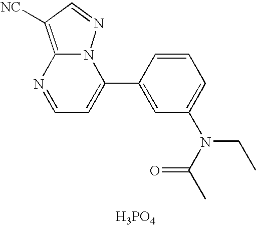

Zaleplon free base (5 g) was dissolved in anhydrous dichloromethane (15 ml), then 1.8 g of 85% of phosphoric acid was added dropwise with stirring. 200 mL of diethyl ether was added to the mixture and this was stirred for 0.5 h. The resulting white solid was filtered, washed with ether and dried in vacuo. Zaleplon phosphate was isolated as a white solid (6.329 g, 95% yield, 100% purity by HPLC).

¹H NMR (400 MHz, DMSO-d6) δ ppm 9.78 (broad, 2 H), 8.92 (d, J=4.49 Hz, 1 H), 8.87 (s, 1 H), 8.09 (d, J=7.61 Hz, 1 H), 8.04 (s, 1 H), 7.57-7.74 (m, 3 H), 3.71 (q, J=7.03 Hz, 2 H), 1.81 (s, 3 H), 1.04 (t, J=6.93 Hz, 3 H).). ¹³C NMR (DMSO-d6) δ ppm 169.24, 154.47, 151.79, 148.04, 147.35, 143.37, 132.07, 131.41, 130.65, 130.36, 129.64, 114.14, 111.66, 82.18, 43.85, 23.47, 13.66. Elemental analysis: C 50.8%, N 17.3%, H 4.5%, O 20.0%.

### EXAMPLE 5

### Differential Scanning Calorimetry (DSC) of Zaleplon and Zaleplon Salts

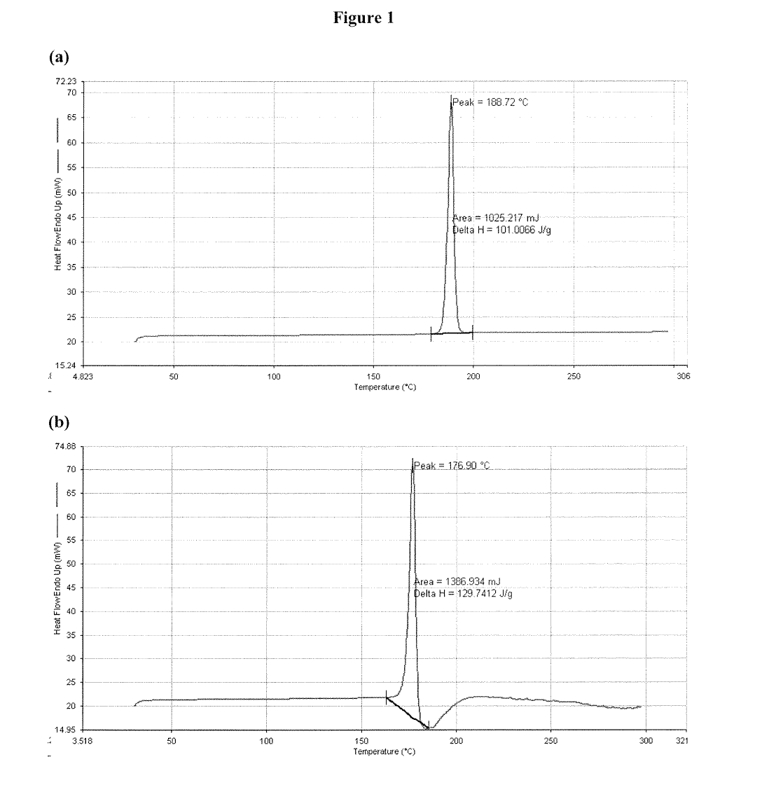

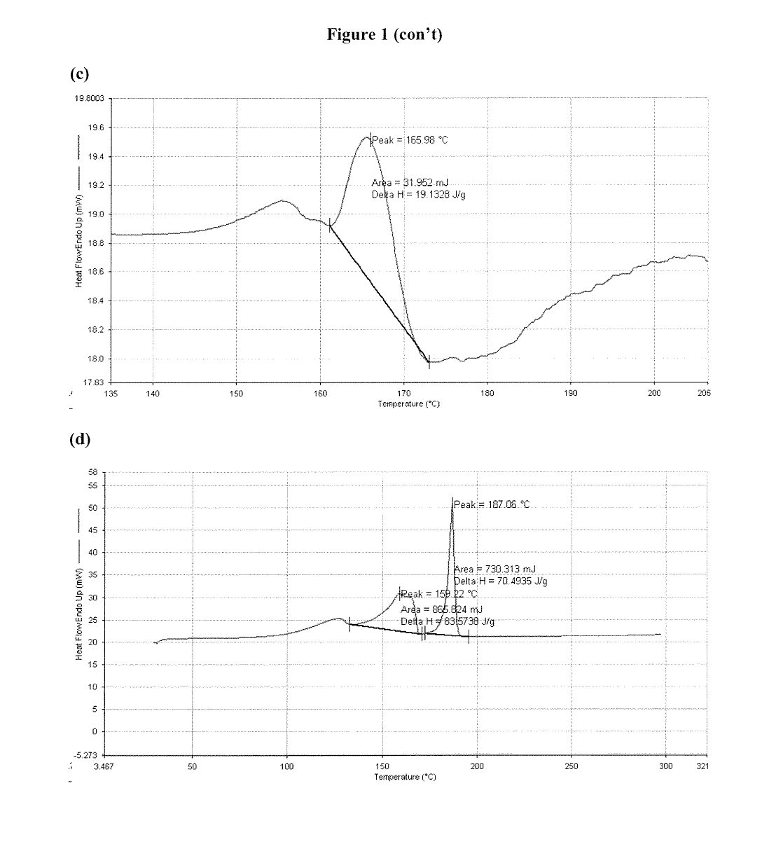

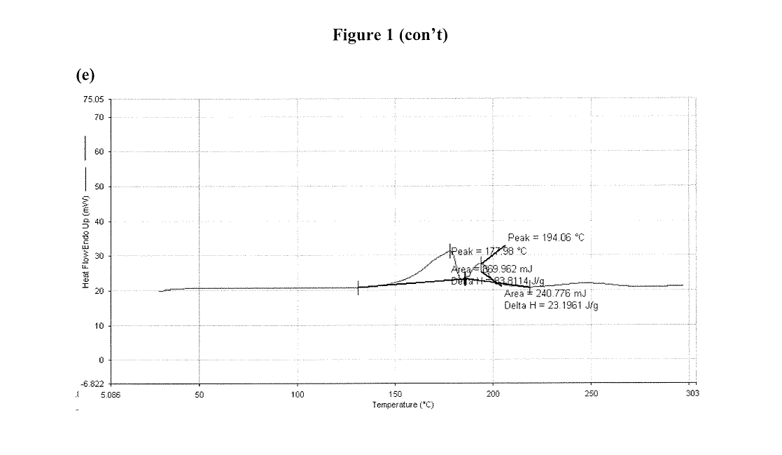

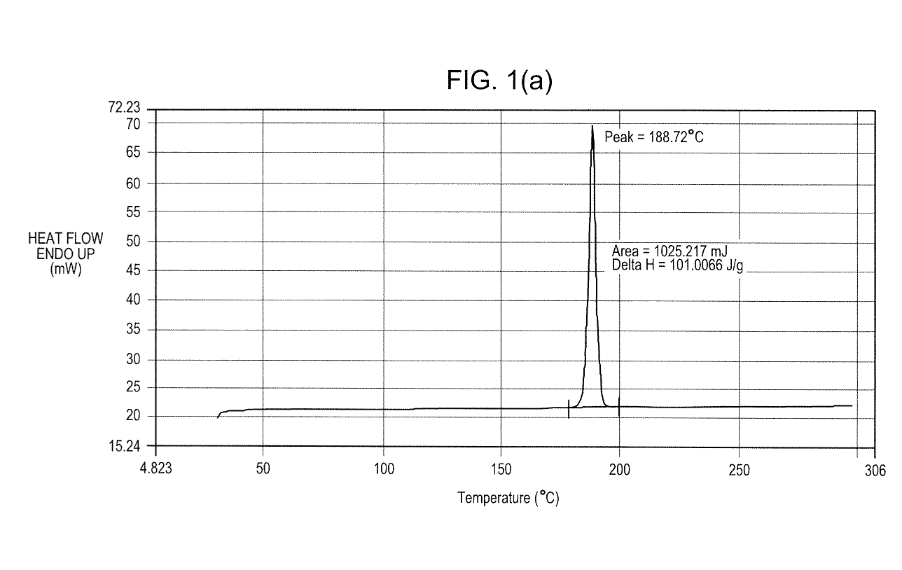

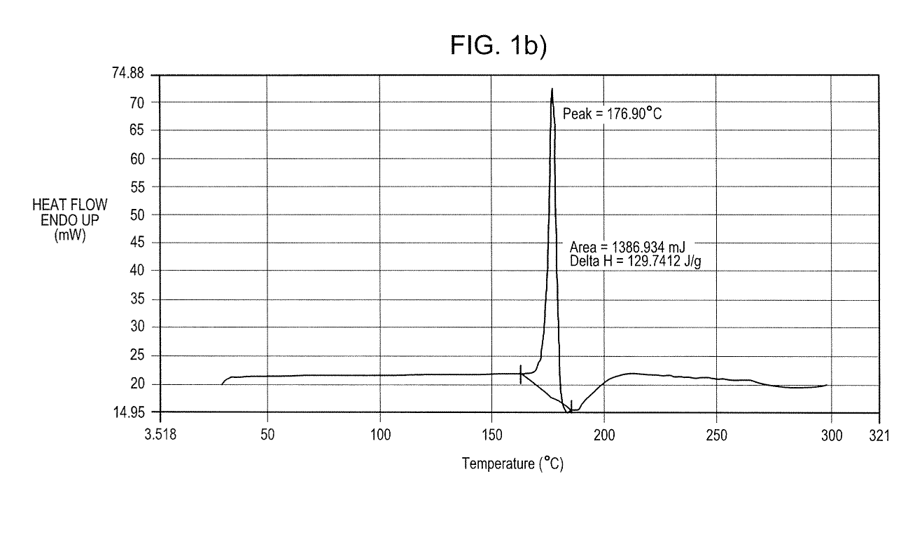

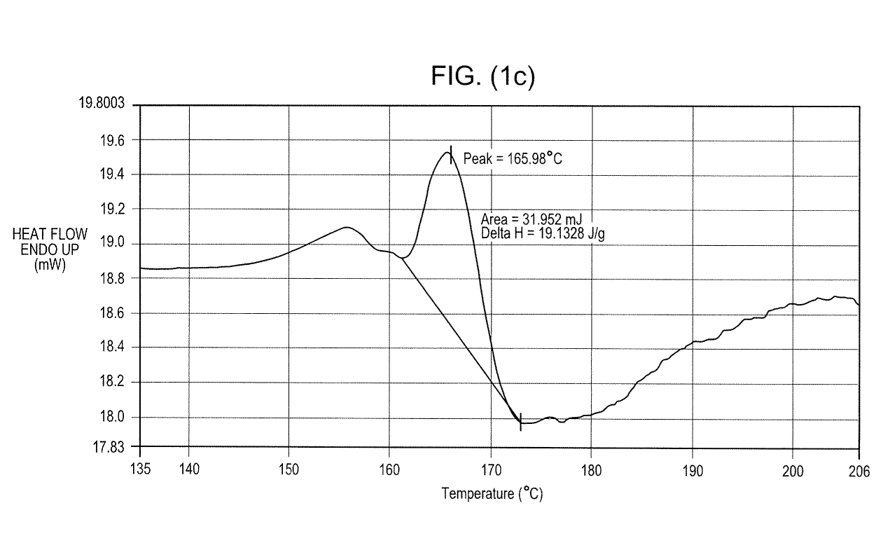

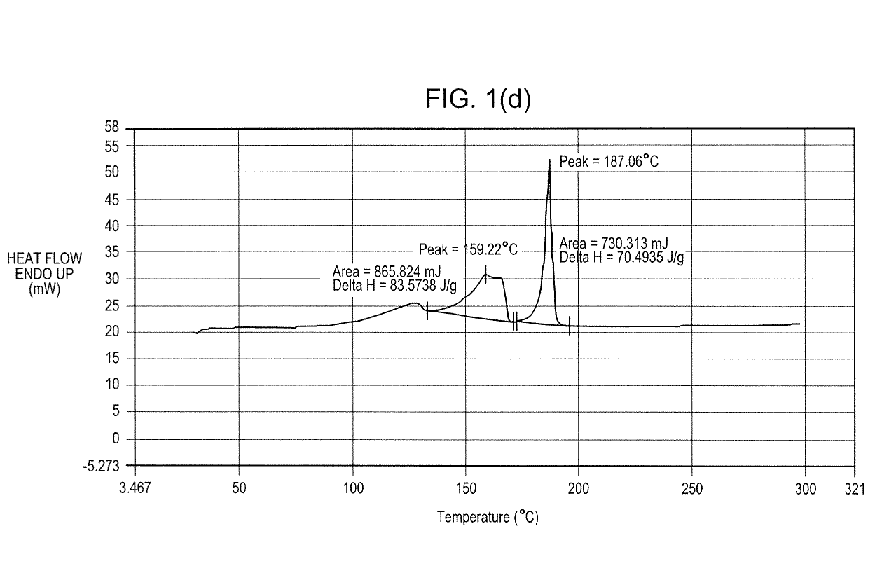

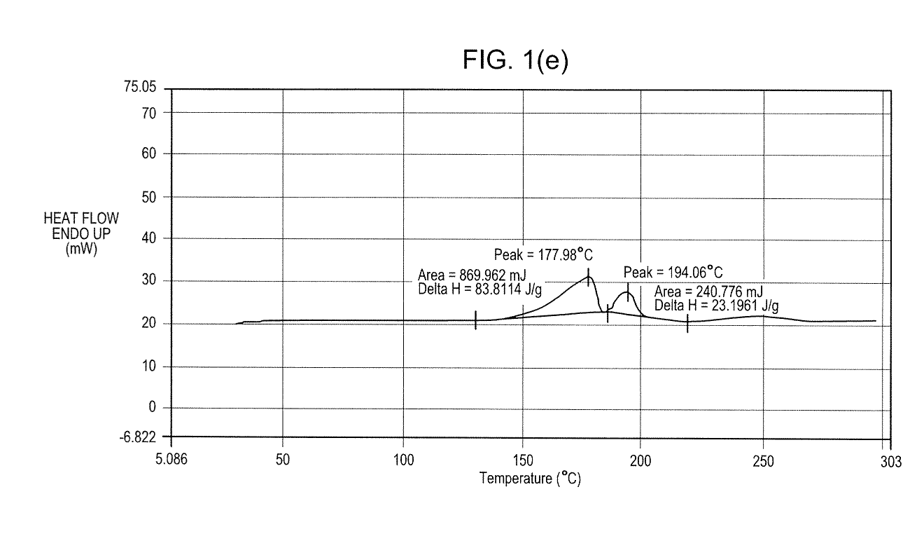

DSC was performed for zaleplon and zaleplon salts. Zaleplon free base melted at 189° C. Zaleplon phosphate was stable and melted at ˜177° C.; some intramolecular exothermic reactions with P(V) were observed at 180° C., wherein P(V) is phosphorus in valency 5. Zaleplon sulfate melted at ˜157° C. and decomposed at 166° C.; some intramolecular redox exothermic reactions with S(IV) were observed at 170° C. (max at 173° C.), wherein S(IV) is sulfur in valency 4. Zaleplon hydrochloride decomposed by at least 3 exothermic processes below 170° C. (at ˜130° C., 160° C. and 165° C.), as shown in FIG. 1d. While one of these processes could be dehydrochlorination, other processes also exist. The formed free base melted at 187° C. Zaleplon hydrobromide underwent melting at 187° C., intramolecular hydrobromination at 194° C. and some intramolecular rearrangements at higher temperatures. This was shown by additional reaction heats. The DSC profiles of zaleplon free base (FIG. 1a), zaleplon phosphate (FIG. 1b), zaleplon sulfate (FIG. 1c), zaleplon hydrochloride (FIG. 1d) and zaleplon hydrobromide (FIG. 1e) are shown.

### EXAMPLE 6

### Synthesis of N-methyl-N-{3-[3-(thiophene-2-carbonyl)-pyrazolo[1,5-a]pyrimidin-7-yl]-phenyl}acetamide (Indiplon) HBr cocrystal

### A. Synthesis of 3-dimethylamino-1-(Thiophene-2-yl)-prop-2-en-1-one

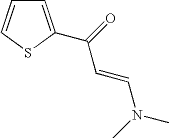

2-Acetylthiophene (500 g, 3.96 moles) was dissolved in dimethylformamide dimethylacetal (755 g, 6.43 moles) and heated to reflux for four hours and forty minutes. The mixture was cooled to room temperature and then poured into heptane (15 L). The brown solid that precipitated was filtered and washed with heptane then dried in vacuo at 50° C. for 4 hour. 3-Dimethylamino-1-thiophene-2-yl-propenone (624 g) was isolated in 87% yield. ¹H NMR (400 MHz, CDCl₃) δ ppm 7.79 (1H, d), 7.63 (1H, d), 7.48 (1H, d), 7.08 (1H, m), 5.62 (1H, d), 3.13 (3H, bs), 2.94 (3H, bs).

### B. Synthesis of 5-(thiophene-2-yl)-isoxazole

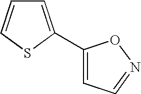

3-Dimethylamino-1-(thiophene-2-yl)-prop-2-en-1-one (624 g, 2.44 moles) and hydroxylamine hydrochloride (239 g, 3.44 moles) were dissolved in methanol (3.4 L). After two hours at reflux the reaction was cooled and the volume reduced under pressure but not concentrated to dryness, since hydroxylamine can be explosive. The mixture was poured into water (800 ml) and extracted with dichloromethane (2×2000 ml). The organic portion was dried over magnesium sulfate, filtered and concentrated under reduced pressure. An orange oil (532 g, 102% yield) was isolated and identified as 5-thiophene-2-yl-isoxazole. ¹H NMR (400 MHz, CDCl₃) δ ppm 8.23 (1H, d), 7.50 (1H, d), 7.42 (1H, d), 7.10 (1H, t), 6.37 (1H, d).

### C. Synthesis of 3-dimethylamino-2-(thiophene-2-carbonyl)acrylonitrile

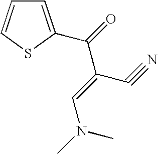

5-Thiophene-2-yl-isoxazole (532 g, 0.2426 moles) and dimethylformamide dimethylacetal (1050 ml) were heated to reflux for three hours. Upon cooling a brown solid precipitated. The mixture was diluted with heptane (3000 ml) and filtered, the filter cake was washed with heptane (2×) and the resulting brown solid was dried in vacuo at 50° C. for 48 hours. 3-Dimethylamino-2-(thiophene-2-carbonyl)acrylonitrile (622 g, 85.8% yield) was identified by NMR. ¹H NMR (400 MHz, CDCl₃) δ ppm 8.21 (1H, d), 8.50 (1H, s), 7.59 (1H, d), 7.12 (1H, t), 3.50 (3H, s), 3.31 (3H, s).

### D. Synthesis of (3-amino-1H-pyrazol-4-yl)-thiophen-2-yl-methanone

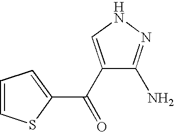

3-Dimethylamino-2-(thiophene-2-carbonyl)acrylonitrile (311 g, 1.50 moles), aminoguanidine nitrate (258 g, 1.88 moles), 10 N sodium hydroxide (266 ml) and ethanol (1662 ml) were combined and heated to reflux for six hours. The mixture was concentrated under reduced pressure and the residue poured into water (6 L). An orange solid precipitated and was filtered, washed with water and ethyl ether and dried in vacuo for twelve hours at 50° C. The material was identified as (3-Amino-1H-pyrazol-4-yl)-thiophen-2-yl-methanone (56 g, 24.6% yield). LC/MS (M+H) 194. ¹H NMR (400 MHz, CDCl₃) δ ppm 8.01 (1H, s), 7.78 (1H, d), 7.64 (1H, d), 7.19.

### E. Synthesis of N-(3-acetylphenyl)-N-methylacetamide

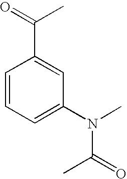

N-(3-Acetylphenyl)acetamide (100 g, 0.564 moles), powdered sodium hydroxide (33.86 g, 0.8465 moles) and toluene (750 ml) were combined and stirred at room temperature for thirty minutes. The reaction mixture was cooled to 0-5° C. and methyl p-toluenesulfonate (115.6 g, 0.6208 moles) was added over one hour. The mixture was heated to 50° C. for twelve hours. The TLC (7:3; ethyl acetate:heptane) showed the reaction was complete. Water (500 ml) was added and the layers separated. The organic layer was washed with saturated aqueous sodium sulfate (500 ml). The organic portion was dried over magnesium sulfate, filtered and concentrated under reduced pressure to a yellow solid (52.6 g). The aqueous portion was reextracted with dichloromethane (3×), dried over magnesium sulfate, filtered and concentrated under reduced pressure to a yellow solid (56.6 g). The two portions found to be the N-(3-acetylphenyl)-N-methylacetamide (100% yield) were combined. LC/MS (M+H) 192. ¹H NMR (400 MHz, CDCl₃) δ ppm 7.90 (1H, d), 7.78 (1H, s), 7.52, (1H, t), 7.40 (1H, d), 3.27 (3H, s), 2.61 (3H, s), 1.86 (3H, s).

### F. Synthesis of N-[(3-dimethylaminoacryloyl)phenyl]-N-methylacetamide

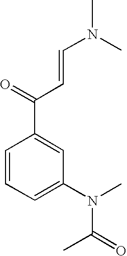

N-(3-Acetylphenyl)-N-methylacetamide (123.6 g, 0.646 moles) and dimethylformamide dimethylacetal (172 ml, 1.29 moles) were combined and heated to reflux. After 5 hours the reaction was complete as determined by TLC (100% ethyl acetate). The reaction was diluted with heptane (350 ml) and the resulting solid filtered and dried in vacuo at room temperature. N-[(3-Dimethylaminoacryloyl)phenyl]-N-methylacetamide (145 g, 91% yield) was isolated as an orange solid.

### G. Synthesis of N-methyl-N-{3-[3-(thiophene-2-carbonyl)-pyrazolo[1,5-a]pyrimidin-7-yl]-phenyl}acetamide HBr cocrystal

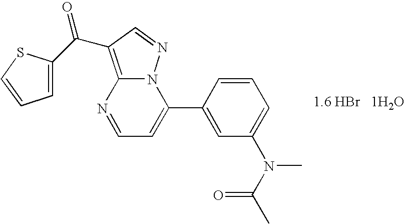

N-[(3-Dimethylaminoacryloyl)phenyl]-N-methylacetamide (129.5 g, 0.52 moles) and (5-amino-1H-pyrazol-4-yl)-thiophen-2-yl-methanone (78.0 g, 0.40 moles) were combined in acetic acid (62.5 ml) and heated to reflux. After 3 hours, TLC (9:1; dichloromethane:methanol) showed the reaction was complete. The cooled reaction mixture was diluted with water (3 L) and the resulting solid filtered and washed with water. The solid was triturated with ethyl acetate (780 ml) for two hours, filtered and dried in vacuo at 50° C. for 48 hours. Isolated N-methyl-N-{3-[3-(thiophene-2-carbonyl)-pyrazolo[1,5-a]pyrimidin-7-yl]-phenyl}acetamide was obtained as a yellow solid in 90% yield and 99% purity. LC/MS (M+H).377 ¹H NMR (400 MHz, CDCl₃) δ ppm 8.86 (1H, d), 8.74 (1H, s), 8.12 (1H, d), 8.00 (2H, s), 7.73 (1H, d), 7.68 (1H, t), 7.49 (1H, d), 7.23 (1H, d), 7.17 (1H, d), 3.37 (3H, s), 2.02 (3H,s). ¹³C NMR (400 MHz, CDCl₃) δ ppm 179.30, 170.41, 152.15, 148.47, 147.5, 146.53, 144.94, 133.50, 131.70, 130.29, 130.06, 128.49, 127.91, 111.17, 109.46, 37.23, 22.65. N-methyl-N-{3-[3-(thiophene-2-carbonyl)-pyrazolo[1,5-a]pyrimidin-7-yl]-phenyl}acetamide (5.2 g) was dissolved in acetic acid (20 ml) and 33% acetic acid/HBr (˜15 ml) was added dropwise. Ethyl ether (50 ml) was added. The resulting solid was filtered, triturated with ethyl acetate, filtered and washed with ethyl acetate. The orange solid was dried in vacuo at 50° C. for 12 hours. ¹H NMR (400 MHz, DMSO) δ ppm 9.38 (2H, bs), 8.91 (1H, d), 8.83 (1H, s), 8.22 (1H, d), 8.09 (1H, s) 8.06 (1H, d), 7.60 (3H, m), 7.30 (1H, t), 3.24 (3H, s), 2.02 (3H, s). ¹³C NMR (400 MHz, DMSO) δ ppm 178.70, 172.08, 169.26, 153.057, 147.80, 147.20, 146.18, 133.40, 129.92, 128.68, 110.54, 110.08, 22.58, 111.17, 21.19. Elemental analysis as calculated for C₂₀H₁₆N₄O₂S₁.1.6 HBr.1H₂O: C, 45.85; H, 3.77; Br, 24.40. Elemental analysis as found: C, 46.07; H, 3.93; Br, 24.11.

### EXAMPLE 7

### Synthesis of Pyridin-2-yl(7-(pyridin-4-yl)pyrazolo[1,5-a]pyrimidin-3-yl)methanone Salts

Using methods similar to those described in Examples 1-3 above, various pyridin-2-yl(7-(pyridin-4-yl)pyrazolo[1,5-a]pyrimidin-3-yl)methanone (ocinaplon) salts were prepared from ocinaplon free base. In particular, ocinaplon di HCl was prepared according to the method described in Example 1, ocinaplon di HBr was prepared according to the method described in Example 2 and ocinaplon disulfate was prepared according to the method described in Example 3. Analytical results for each of the ocinaplon salts so produced are shown below.

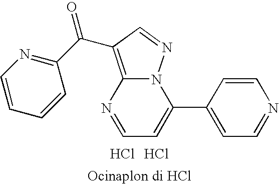

¹H NMR (400 MHz, METHANOL-d₄) δ ppm 6.28 (d, J=4.49 Hz, 1 H) 6.67-6.74 (m, 1 H) 7.17-7.23 (m, 2 H) 7.28-7.34 (m, 2 H) 7.39-7.45 (m, 2 H) 7.47 (d, J=5.47 Hz, 1 H) 7.59 (d, J=6.83 Hz, 2 H).

¹³C NMR (400 MHz, CHLOROFORM-d) δ ppm 114.20 129.45 129.63 131.02 143.92 144.16 144.88 148.01 148.36 148.46 150.01 150.26 155.82 180.03

LC/MS (M+1) 301.8.

DSC (30-450° C., 25° C./min) 209.33° C., 232.99° C.

TGA (ambient-500° C., 10° C./min) weight loss of 12.04% until 160° C., weight loss of another 10.18% until 203° C., weight loss of another 66.82% until 364° C., weight loss of another 2.89% until 472° C.

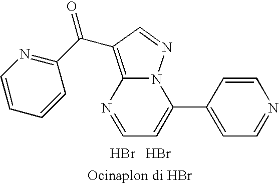

¹H NMR (400 MHz, DEUTERIUM OXIDE) δ ppm 7.80 (d, J=4.49 Hz, 1 H) 8.25 (t, J=6.74 Hz, 1 H) 8.62 (d, J=7.81 Hz, 1 H) 8.69-8.78 (m, 3 H) 8.89 (s, 1 H) 8.99 (d, J=5.47 Hz, 1 H) 9.05 (d, J=4.49 Hz, 1 H) 9.09 (d, J=6.44 Hz, 2 H).

¹³C NMR (400 MHz, DEUTERIUM OXIDE) δ ppm 108.80 113.59 128.08 128.15 130.05 142.51 143.38 144.13 146.49 146.81 148.91 149.31 155.52 180.59

Anal. Calcd for: C₁₇H₁₃Br₂N₅O_{1:} C, 44.09; H, 2.83; Br, 34.51; N, 15.12; O 3.45. Found: C, 42.46; H, 3.12; Br, 32.95; N, 14.44; O, 6.39. LC/MS (M+1) 301.8

DSC (30-450° C., 25° C./min) 167.80° C., 213.32° C.

TGA (ambient-500° C., 10° C./min) weight loss of 3.51% until 120° C., weight loss of another 3.25% until 193° C., weight loss of another 31.14% until 287° C., weight loss of another 40.21% until 362° C., weight loss of another 3.84% until 481° C.

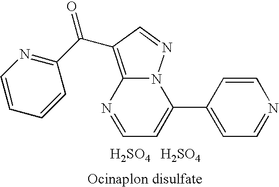

¹H NMR (400 MHz, METHANOL-d₄) δ ppm 6.26 (d, J=4.30 Hz, 1 H) 6.50-6.56 (m, 1 H) 6.96-7.07 (m, 2 H) 7.29 (d, J=6.83 Hz, 2 H) 7.41 (d, J=5.27 Hz, 1 H) 7.47 (d, J=4.49 Hz, 1 H) 7.56-7.61 (m, 2 H)

¹³C NMR (400 MHz, DMSO-d₆) δ ppm 109.15 112.04 123.74 126.74 127.25 138.79 142.37 144.15 144.47 148.22 148.98 149.19 153.91 154.10 184.19).

Anal. Calcd for C₁₇H₁₅N₅O₉S₂: C, 41.05; H, 4.04; N, 14.08; O 28.95; S, 12.89. Found: C, 39.50; H, 3.33; N, 13.48; O 31.07; S, 12.46. LC/MS (M+1) 301.8

DSC (30-350° C., 25° C./min) 108.55° C., 266.51° C.

TGA (ambient-500° C., 10° C./min) weight loss of 3.80% until 160° C., weight loss of another 3.410% until 273° C., weight loss of another 45.42% until 484° C.

### EXAMPLE 8

### Equilibrium Aqueous Solubility of Solid Ocinaplon Salts

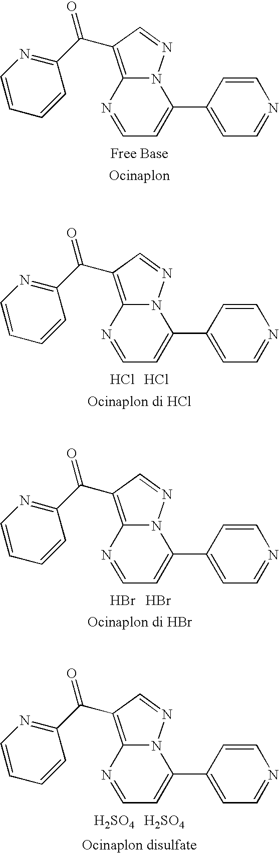

Samples of the ocinaplon salts shown above were evaluated for equilibrium aqueous solubility from solid. Duplicate samples of ocinaplon were accurately weighed and diluted to 1 ml with water to 6.83 mg/ml and 6.43 mg/ml. Duplicate samples of Ocinaplon di HCl salt were accurately weighed and diluted to 1 ml with water to 5.5 mg/ml and 6.0 mg/ml. Ocinaplon di HBr salt was accurately weighed and diluted to 1 ml with water to 6.1 mg/ml and 5.8 mg/ml. Ocinaplon disulfate salt was accurately weighed and diluted to 1 ml with water to 7.5 mg/ml and 6.5 mg/ml. Any change in pH of the water following sample addition was recorded. After equilibration at room temperature overnight the solutions were filtered and solute concentration determined by fast gradient HPLC with UV/VIS/MS detection with reference to 5, 10, 50, 100 and 250 μM analytical standards. Prior to sample filtration, filters were primed with 600 μl of sample to resolve potential adsorption problems. Both sample replicates were collected through the primed filter. Duplicate determinations were made in all cases. The results of this study are shown in Table 1.

TABLE 1
Equilibrium Aqueous Solubility of Solid Ocinaplon Salts

| | |
|---|---|
| | Equilibrium Aqueous |
| Compound | Solubility from Solid |

| | | |
|---|---|---|
| Ocinaplon | H₂0 | 0.4 mM (0.1 mg/ml) |
| Free base | | |
| Ocinaplon | H₂0 | 12.6 mM (4.7 mg/ml) |
| di HCl salt | | |
| Ocinaplon | H₂0 | 11.5 mM (5.3 mg/ml) |
| di HBr | | |
| Ocinaplon | H₂0 | 10.6 mM (5.3 mg/ml) |
| disulfate salt | | |

### EXAMPLE 9

### Pharmacokinetic Parameters of Indiplon Compared to Indiplon HBr Co-Cyrstal

The purpose of this study was to determine the pharmacokinetics of indiplon following oral administration of the free base and the HBr co-crystal in a suspension to male beagle dogs.

A. Materials and Methods

1. Test Compounds

The test compounds used in the study were the free base and HCl salt of indiplon. The compounds were stored at approximately 2-8° C.

2. Animals

Five male purebred beagles were transferred to the study from a stock colony. Each animal was identified with an individually numbered ear tattoo. The animals were acclimated for 5 days. At the time of dose administration, animals were young adult/adult age. During acclimation and the test period, animals were individually housed in wire-mesh enclosures with coated rod-bottom floors. Animals were not comingled for at least 24 hours after each dose administration to allow monitoring of any test compound-related effects. Animals had access to non-certified Canine Diet #5L03 (PMI Feeds, Inc.) ad libitum, except as specified for dose administration.

3. Dose Preparation

The test compounds were prepared in 0.5% methylcellulose. The target concentrations of the free base and HBr salt were 1.0 and 1.34 mg/mL (equivalent to 1 mg of the free base), respectively. The liquid suspensions were administered via oral gavage. Prior to removing the gavage tube, the tube was flushed with approximately 5 mL of water.

4. Sample Collection and Analysis

Blood (approximately 2 mL) was collected from a jugular vein via syringe and needle and transferred into tubes containing K₂EDTA anticoagulant predose and at 0.5, 1, 2, 3, 4, 6, 8, 12, and 24 hours postdose. Blood was maintained on wet ice, in chilled Kryoracks, or at approximately 5° C. prior to centrifugation to obtain plasma. Centrifugation began within 1 hour of collection. Plasma was placed on dry ice prior to storage at approximately −70° C. Plasma samples, remaining test compounds, and remaining dose formulations were shipped on dry ice to the analysis site and stored at approximately −70° C. before and after analysis. Samples were analyzed using an LC/MS/MS method. Plasma concentration-time data for each dog and formulation were analyzed using a noncompartmental model assumption with WinNonlin, version 4.1 (Pharsight Corporation, Mountain View, Calif.). All calculations were based on the nominal times as actual sample collection times departed minimally from the schedule times.

B. Results

Using the methods described above, indiplon HBr produced a pharmaceutical profile superior to the corresponding free base in several dimensions in 5 out of 5 test animals (100%), including a 69.7% increase in the maximum plasma concentrations achieved (Cmax) and an increase in the AUC of more than two-fold (226.8%), as shown in Table 2 below. Since the pharmacological effect of a compound is generally related to plasma concentration, these data indicate that a lower dose of the HBr co-crystal can be administered to achieve a plasma concentration equal to the free base.

TABLE 2
Pharmacokinetic Parameters of Indiplon in Male Dogs Following a Single
Oral Dose (5 mg Free Base/kg) of the Free Base or HBr co-crystal
in a 0.5% Methylcellulose Suspension).

| | | Cₘₐₓ | Tₘₐₓ | AUC₀₋ₜ |
|---|---|---|---|---|
| Formulation | Dog | (ng/mL) | (hr) | (ng · hr/mL) |
| Free Base | CTZBCG | 266 | 0.500 | 408 |
| Suspension | CUPBEG | 106 | 1.00 | 160 |
| | CUVBAT | 101 | 0.500 | 125 |
| | CVRCVX | 212 | 0.500 | 279 |
| | CVSCCL | 240 | 0.500 | 277 |
| | Mean | 185 | 0.600 | 250 |
| | SD | 76.8 | 0.224 | 112 |
| | CV % | 41 | 37 | 44.9 |
| HBr Salt | CTZBCG | 513 | 0.500 | 1130 |
| Suspension | CUPBEG | 253 | 0.500 | 429 |
| | CUVBAT | 309 | 0.500 | 291 |
| | CVRCVX | 227 | 0.500 | 258 |
| | CVSCCL | 270 | 2.00 | 725 |
| | Mean | 314 | 0.800 | 567 |
| | SD | 115 | 0.671 | 365 |
| | CV % | 37 | 84 | 64.5 |

In the table, each unique set of letters refers to an individual animal; this study was a N = 5, cross-over design using the same dogs for all conditions, thereby allowing a direct comparison of the effect of the salt and free base in a single animal.

### EXAMPLE 10

### Pharmacokinetic Parameters of Different Salt Forms of Ocinaplon in Male Beagle Following a Single Oral Dose

The pharmacokinetic parameters of different pyrazolopyrimidine acid salts vary in an apparently salt and compound dependent fashion. This is illustrated by a comparison of the pharmacokinetic properties of different acid salts of the pyrazolopyrimidine, ocinaplon. This study was performed in a manner similar to the study described in Example 9 hereinabove. The concentrations of the HCl, HBr and disulfate salts of ocinaplon were 3.72 mg/mL, 4.61 mg/mL and 4.95 mg/mL, respectively. All concentrations were calculated to be 3.0 mg free base/mL; the dose volume was 10 mg/kg. The total dose was 30 mg free base/kg. As shown in Table 3, a comparison of the effects of the diHCl, diHBr, and diH₂S0₄ salts of ocinaplon to the free base (all compounds administered as a suspension in 0.5% methylcellulose) revealed that the Cmax produced by each of these salts exceeded by more than two-fold the Cmax of ocinaplon produced by administering an equivalent amount of the free base (administered in micronized form to facilitate maximum absorption). In addition, a lower variability in plasma levels of ocinaplon was produced by administering the HCl and HBr salts but not the sulfate salt of ocinaplon. The AUC of ocinaplon produced by administration of the HCl salt was almost twice as high as that produced by an equivalent amount of the free base, and there was much less variability in the AUC when administered as the HCl form compared to the free base, as shown in Table 3 by the lower CV % value for the HCl form compared to the free base. While the HBr salt resulted in Cmax values of ocinaplon approximately twice those achieved by administering an equivalent amount of free base, the AUC did not differ (i.e., 38000 ng·hr/ml versus 37570 ng·hr/ml). Nonetheless, the variability in AUC observed with the diHBr salt was dramatically less than that produced by the free base. These findings exemplify and underscore the differences in pharmacokinetic profiles among these three exemplary salts of ocinaplon.

TABLE 3
Mean Pharmacokinetic Parameters (with CV % in parentheses) of
Ocinaplon in Male Beagle Dogs Following a Single Oral Dose of Different Salt
Forms of Ocinaplon. This study was a N = 5 cross-over design using the same test
animals for all conditions, thereby allowing a direct comparison of the effect
of the salt and free base in a single test animal.

| | Compound | | | | |
|---|---|---|---|---|---|
| | Di-HCl | Di-HBr | DI-Sulfate | Micronized Free Base | |
| | Salt | Salt | Salt | (0.5% MC) | |
| | Dosage | | | | |
| | (30 mg | (30 mg | (30 mg | | (30 |
| | Base/kg) | Base/kg) | Base/kg) | 50 mg/kg | mg/kg) |
| Cₘₐₓ (ng/mL) | 15000 (19) | 13600 (19) | 14800 (34) | 10410 (25) | 6246 |
| Tₘₐₓ (hr) | 1.10 (50) | 0.90 (46) | 0.80 (34) | 1.80 (15) | — |
| AUC₀₋ₜ (ng · hr/mL) | 57100 (18) | 38000 (21) | 46300 (46) | 62520 (48) | 37570 |
| AUC_{0-∞} | 57300 (18) | 38100 (21) | 46400 (46) | 63180 (46) | 37910 |
| (ng · hr/mL) | | | | | |
| Half-life (hr) | 1.05 (29) | 0.83 (18) | 0.95 (18) | 1.61 (22) | — |

### EXAMPLE 11

### Comparison of the Pharmacokinetic Profile of Zaleplon with its HCl Salt

A comparison of the pharmacokinetic profile of zaleplon with its HCl salt demonstrated important differences, a number of which would be highly desirable for a rapid acting hypnotic agent used to treat patients with insomnia who have difficulty falling asleep. This study was performed in a manner similar to the study described in Example 9 hereinabove. The dose of the free base formulation was 5.0 mg/kg (1.0 mg/mL; 5.0 mL/kg); the dose of the HCl suspension was 2.80 mg/kg (1.12 mg HCl salt/mL ([1.0 mg free base/mL], 2.5 mg/kg). As shown in Table 4, while the Cmax values are essentially equivalent (when calculated on a mg/kg dosing basis), the Tmax value (the time to achieve peak plasma concentration) of the HCl salt is about half that of the free base (that is 1.1 h with the free base versus 0.55 hr with the HCl salt). Another notable difference between the free base and the HCl salt is that the half-life of the HCl salt is reduced by about 27% when compared with the free base, as shown in Table 4 by the lower half-life value for the HCL salt as compared to the free base. The former measure indicates zaleplon HCl would reach therapeutic levels in half the time of the free base, while the latter measure indicates it would be eliminated more rapidly. Both of these features may be desirable features of a drug used to treat patients who have difficulty falling asleep (shorter Tmax), but do not want a “hangover” effect associated with residual drug (shorter half-life). By contrast, the Tmax of indiplon HBr is not remarkably different from the free base, while the Tmax of each of the three ocinaplon salts appears shorter than that of the free base.

TABLE 4
Plasma Pharmacokinetic Comparison of
Zaleplon in Male Beagle Dogs
Following Single Oral Doses of 5.0 mg
Free Base/kg and 2.5 mg HCl Salt/kg

| | | | | AUC₀₋ₜ | AUC_{0-∞} | |
|---|---|---|---|---|---|---|
| | | Cₘₐₓ | Tₘₐₓ | (ng · | (ng · | Half-life |
| | Dog No. | (ng/mL) | (hr) | hr/mL) | hr/mL) | (hr) |
| Free Base | CLT-2 | 1850 | 1.00 | 6840 | 6860 | 1.15 |
| (5 mg/kg) | H0M004 | 1460 | 1.00 | 6930 | 7000 | 1.53 |
| | H1B108 | 926 | 2.00 | 3760 | 3760 | 0.774 |
| | TDR2 | 1100 | 0.500 | 2550 | 2550 | 1.24 |
| | TKR-2 | 2630 | 1.00 | 13400 | 14500 | 3.02 |
| | Mean | 1590 | 1.10 | 6700 | 6940 | 1.54 |
| | SD | 678 | 0.548 | 4220 | 4660 | 0.869 |
| | CV % | 43 | 50 | 63 | 67 | 56 |
| HCl Salt | CLT-2 | 1010 | 0.250 | 1820 | 1820 | 0.756 |
| (2.5 | H0M004 | 642 | 0.500 | 2010 | 2010 | 1.20 |
| mg/kg) | H1B108 | 587 | 1.00 | 1360 | 1360 | 0.862 |
| | TDR2 | 820 | 0.500 | 2650 | 2650 | 1.12 |
| | TKR-2 | 896 | 0.500 | 3720 | 3760 | 1.70 |
| | Mean | 790 | 0.550 | 2310 | 2320 | 1.13 |
| | SD | 175 | 0.274 | 913 | 927 | 0.368 |
| | CV % | 22 | 50 | 40 | 40 | 33 |

The data in Examples 8 through 11 show that certain pharmaceutically acceptable salts and co-crystals of pyrazolopyrimidines confer distinct and important advantages as drugs compared to the free base.

It will be understood that the instant invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

All publications and patents mentioned herein are incorporated herein by reference for the purpose of describing and disclosing, for example, the constructs and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### REFERENCES

U.S. Pat. No. 4,521,422; Jun. 4, 1985; Dusza et al.
U.S. Pat. No. 6,399,621; Jun. 4, 2002; Dusza et al.
U.S. Pat. No. 6,384,221; May 7, 2002; Thiele et al.
U.S. Patent Application Publication, Publication No. 2002/0198221, Dec. 22, 2002, Dusza et al.
Vanover, K. E. et al., Discriminative Stimulus and Anxiolytic-Like Effects of the Novel Compound CL 273,547, Experimental and Clinical Psychopharmacology, Vol. 2, No. 3, 223-233, 1994
Damgen, K. and Luddens, H., Zaleplon displays a selectivity to recombinant GABA_{A} receptors different from zolpidem, zopiclone and benzodiazepines, Neurosci. Res. Commun. 25:139-148, 1999
Sanna, E. et al, Comparison of the effects of zaleplon, zolpidem and triazolam atvarious GABA(A) receptor subtypes, Eur. J. Pharmacol. 451:103-110, 2002
Petroski, R. E. et al., Indiplon is a High-Affinity Positive Allosteric Modulator with Selectivity for α1 Subunit-Containing GABA_{A} Receptors, The Journal of Pharmacology and Experimental Therapeutics, Vol. 317, No. 1, 369-377, 2006
Lippa, A. et al., Selective anxiolysis produced by ocinaplon, a GABA_{A} receptor modulator, PNAS, Vol. 102, No. 20, 7380-7385, 2005
Barnard, E. A. et al., International Union of Pharmacology. XV. Subtypes of γ-Aminobutyric AcidA Receptors: Classificaton on the Basis of Subunit Structure and receptor Function, Pharmacological Reviews, Vol. 50, No. 2, 291-313, 1998
Roth, T. et al., Efficacy and tolerability of indiplon (NBI 34060) solution in healthy adults in a model of transient insomia, Sleep 26:A87, 2003
Jochelson, P. et al., Lack of pharmacological and pharmacokinetic tolerance following repeat doses of indiplon (NBI 34060), Sleep 26:A85, 2003
Shen, J. et al., Synthesis of Zaleplon, Zhongguo Yiyao Gongye Zazhi, Vol. 33, No, 7, pp. 313-314, 2002.

What is claimed is:

1. A pharmaceutically acceptable salt or co-crystal of a compound of the pyrazolopyrimidine class, wherein the compound is N-[3-(3-cyanopyrazolo[1,5-a]pyrimidin-7-yl)phenyl]-N-ethylacetamide, N-methyl-N-{3-[3-(thiophene-2-carbonyl)-pyrazolo[1,5-a]pyrimidin-7-yl]-phenyl}acetamide or pyridin-2-yl(7-(pyridin-4-yl)pyrazolo[1,5-a]pyrimidin-3-yl)methanone.

2. The pharmaceutically acceptable salt or co-crystal according to claim 1, wherein the compound is N-[3-(3-cyanopyrazolo[1,5-a]pyrimidin-7-yl)phenyl]-N-ethylacetamide.

3. The pharmaceutically acceptable salt or co-crystal according to claim 1, wherein the compound is N-methyl-N-{3-[3-(thiophene-2-carbonyl)-pyrazolo[1,5-a]pyrimidin-7-yl]-phenyl}acetamide.

4. The pharmaceutically acceptable salt or co-crystal according to claim 1, wherein the compound is pyridin-2-yl(7-(pyrimidin-4-yl)pyrazolo[1,5-a]pyrimidin-3-yl)methanone.

5. The pharmaceutically acceptable salt or co-crystal according to claim 2, wherein the salt is the hydrochloride, hydrobromide, sulfate or phosphate salt.

6. The pharmaceutically acceptable salt or co-crystal according to claim 3, wherein the salt is the hydrobromide or sulfate salt.

7. The pharmaceutically acceptable salt or co-crystal according to claim 4, wherein the salt is the hydrochloride, hydrobromide, phosphate or sulfate salt.

8. The pharmaceutically acceptable salt or co-crystal according to claim 4, wherein the salt is the dihydrochloride, dihydrobromide, diphosphate or disulfate salt.

9. The pharmaceutically acceptable salt or co-crystal of a compound of the pyrazolopyrimidine class according to claims 2, 3 or 4 having one or more improved pharmacokinetic properties when compared to the flee base of the compound of the pyrazolopyrimidine class.

10. The pharmaceutically acceptable salt or co-crystal according to claim 9, wherein the improved pharmacokinetic properties are selected from the group consisting of increased plasma concentration summed over time, higher maximum plasma concentration, decreased time to peak drug concentration in plasma, reduced plasma half-life and decreased decreased inter- and intra-subject variability in plasma concentration summed over time.

11. A composition comprising a pharmaceutically acceptable salt or co-crystal of a compound of the pyrazolopyrimidine class, wherein the compound is N-[3-(3-cyanopyrazolo[1,5-a]pyrimidin-7-yl)phenyl]-N-ethylacetamide, N-methyl-N-{3-[3-(thiophene-2-carbonyl)-pyrazolo[1,5-a]pyrimidin-7-yl]-phenyl}acetamide or pyridin-2-yl(7-(pyridin-4-yl)pyrazolo[1,5-a]pyrimidin-3-yl)methanone.

12. The composition according to claim 11 wherein the compound is N-[3-(3-cyanopyrazolo[1,5-a]pyrimidin-7-yl)phenyl]-N-ethylacetamide.

13. The composition according to claim 11 wherein the compound is N-methyl-N-{3-[3-(thiophene-2-carbonyl)-pyrazolo[1,5-a]pyrimidin-7-yl]-phenyl}acetamide.

14. The composition according to claim 11 wherein the compound is pyridin-2-yl(7-(pyridin-4-yl)pyrazolo[1,5-a]pyrimidin-3-yl)methanone.

15. The composition according to claim 11 further comprising a pharmaceutically acceptable carrier.

16. A method for preparing a pharmaceutically acceptable salt or co-crystal of a compound of the pyrazolopyrimidine class, wherein the compound is N-[3-(3-cyanopyrazolo[1,5-a]pyrimidin-7-yl)phenyl]-N-ethylacetamide, N-methyl-N-{3-[3-(thiophene-2-carbonyl)-pyrazolo[1,5-a]pyrimidin-7-yl]-phenyl}acetamide or pyridin-2-yl(7-(pyridin-4-yl)pyrazolo[1,5-a]pyrimidin-3-yl)methanone, comprising reacting the compound with a salt-forming acid.

17. The method according to claim 16, wherein the compound is N-[3-(3-cyanopyrazolo[1,5-a]pyrimidin-7-yl)phenyl]-N-ethylacetamide and the salt-forming acid is hydrochloric acid, hydrobromic acid, sulfuric acid or phosphoric acid.

18. The method according to claim 16, wherein the compound is N-methyl-N-{3-[3-(thiophene-2-carbonyl)-pyrazolo[1,5-a]pyrimidin-7-yl]-phenyl}acetamide and the salt-forming acid is hydrobromic acid or sulfuric acid.

19. The method according to claim 16, wherein the compound is pyridin-2-yl(7-(pyridin-4-yl)pyrazolo[1,5-a]pyrimidin-3-yl)methanone and the salt-forming acid is hydrochloric acid, hydrobromic acid, phosphoric acid or sulfuric acid.

20. The method according to claim 16 further comprising isolating the pharmaceutically acceptable salt or co-crystal of a compound of the pyrazolopyrimidine class.

21. The method according to claim 20 wherein the pharmaceutically acceptable salt or co-crystal of a compound of the pyrazolopyrimidine class is isolated by first precipitating and then collecting the pharmaceutically acceptable salt or co-crystal.

22. The method according to claim 21 wherein the pharmaceutically acceptable salt or co-crystal of a compound of the pyrazolopyrimidine class is precipitated by the addition of a precipitant.

23. The method according to claim 21 wherein the pharmaceutically acceptable salt or co-crystal of a compound of the pyrazolopyrimidine class is collected by filtration.

24. A method for treating a disease or condition of the central nervous selected from the group consisting of a sleep disorder, an anxiety disorder, a seizure disorder, muscle spasms, tinnitus, pain and acute psychosis comprising administering to a mammalian subject in need of such treatment a therapeutically effective amount of a pharmaceutically acceptable salt or co-crystal of a compound of the pyrazolopyrimidine class, wherein the compound is N-[3-(3-cyanopyrazolo[1,5-a]pyrimidin-7-yl)phenyl]-N-ethylacetamide, N-methyl-N-{3-[3-(thiophene-2-carbonyl)-pyrazolo[1,5-a]pyrimidin-7-yl]-phenyl}acetamide or pyridin-2-yl(7-(pyridin-4-yl)pyrazolo[1,5-a]pyrimidin-3-yl)methanone.

25. The method according to claim 24 wherein the compound is N-[3-(3-cyanopyrazolo[1,5-a]pyrimidin-7-yl)phenyl]-N-ethylacetamide.

26. The method according to claim 24 wherein the compound is N-methyl-N-{3-[3-(thiophene-2-carbonyl)-pyrazolo[1,5-a]pyrimidin-7-yl]-phenyl}acetamide.

27. The method according to claim 24 wherein the compound is pyridin-2-yl(7-(pyridin-4-yl)pyrazolo[1,5-a]pyrimidin-3-yl)methanone.

28. The method according to claim 24 wherein the sleep disorder is insomia.

29. The method according to claim 24 wherein the seizure disorder is epilepsy.
